(19) United States
(12) Patent Application Publication
(10) Pub. No.: US 2022/0152256 A1
(43) Pub. Date: May 19, 2022

(54) Sanitizing Steamer

(71) Applicant: Bofill Holdings LLC, Opa Locka, FL (US)

(72) Inventor: Peter Bofill, Opa Locka, FL (US)

(21) Appl. No.: 16/953,243

(22) Filed: Nov. 19, 2020

(51) Int. Cl. A61L 2/26; A61L 2/10; D06F 39/00

(52) U.S. Cl. CPC A61L 2/26; A61L 2/10; A61L 2202/26; A61L 2202/11; A61L 2202/17; D06F 39/008

(57) ABSTRACT

A steamer with sanitization means having a steam unit, a sanitization unit, and a shroud. The steam unit includes a reservoir, a handle, and a nozzle, wherein the nozzle is in fluid communication with the nozzle. The sanitization unit includes a light assembly and an actuation mechanism. The light assembly is configured to emit sterilizing wavelengths of light, such as ultraviolet light, and is operably coupled to the actuation mechanism, such that when the actuation mechanism is triggered, the light assembly is switched from an off state to an on state. The light assembly is integrated about the nozzle in order to emit light as steam is dispensed from the nozzle. The shroud is removably attached to the nozzle and positioned about the light assembly, such that the light is concentrated to the area of the shroud. The shroud may also be collapsible to enhance portability.

### CROSS-REFERENCES TO RELATED APPLICATIONS

None.

### STATEMENT REGARDING FEDERAL SPONSORED RESEARCH OR DEVELOPMENT

None.

### NAMES OF PARTIES TO A JOINT RESEARCH AGREEMENT

None.

### REFERENCE TO A “SEQUENCE LISTING”, A TABLE, OR A COMPUTER PROGRAM LISTING APPENDIX SUBMITTED ON COMPACT DISC AND INCORPORATION-BY-REFERENCE OF THE MATERIAL ON THE COMPACT DISCLOSURE

None.

### STATEMENT REGARDING PRIOR DISCLOSURES BY AN INVENTOR OR JOINT INVENTOR

None.

### BACKGROUND OF THE INVENTION

### 1. Field of the Invention

The present invention relates to fabric cleaners, and more particularly, to an improved device and method for steam cleaning and sanitizing.

### Description of Related Art Including Information Disclosed under 37 CFR 1.97 And 37 CFR 1.98

Several designs for steamers have been designed in the past. None of them, however, include means for sterilizing fabrics. While traditional steamers are able to remove wrinkles and odors and possibly loosen some debris on fabric, they are not able to kill germs, bacteria, and viruses. More specifically, traditional steamers do not include a means for sanitizing fabrics with irradiating light or chemicals. Thus, traditional steamers can only partially cleanse a fabric to enhance the odor or appearance.

These traditional steamers do not sterilize the fabric and thus do not help protect the user from harmful substances when contacting the fabric. As such, an additional sanitation means would greatly enhance the use of a steam cleaner by simultaneously enhancing the cosmetics of the fabric and making the fabric safer for human use.

Other patents describing the closest subject matter provide for a number of more or less complicated features that fail to solve the problem in an efficient and economical way. None of these patents suggest the novel features of the present invention.

A brief abstract of the technical disclosure in the specification and title are provided as well for the purposes of complying with 37 CFR 1.72 and are not intended to be used for interpreting or limiting the scope of the claims.

Without limiting the scope of the invention, a brief summary of some of the claimed embodiments of the invention is set forth below. Additional details of the summarized embodiments of the invention and/or additional embodiments of the invention may be found in the detailed description of the invention below.

### BRIEF SUMMARY OF THE INVENTION

It is one of the main objects of the present invention to provide a device for steam cleaning fabric.

It is another object of this invention to provide a steamer with sanitation means, in which the sanitation means includes the use of solvents that are dispensed in conjunction with the steam.

It is still another object of the present invention to provide a steamer with sanitization means, in which the sanitization means includes the use of irradiating light.

It is yet another object of this invention to provide such a steamer with sanitization means that is inexpensive to manufacture and maintain while retaining its effectiveness.

Further objects of the invention will be brought out in the following part of the specification, wherein detailed description is for the purpose of fully disclosing the invention without placing limitations thereon.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE SEVERAL VIEWS OF THE DRAWING

With the above and other related objects in view, the invention exists in the details of construction and combination of parts as will be more fully understood from the following description, when read in conjunction with the accompanying drawings in which:

FIG. 1 shows a front perspective view of a steamer with sanitization means, wherein a light assembly is integrated about a nozzle of a steam unit.

FIG. 2 shows a front perspective view of the steamer with sanitization means, wherein a shroud is attached to the nozzle and positioned around the light assembly.

FIG. 3 shows a front perspective view depicting the removable and collapsible nature of the shroud as indicated by the bold arrows.

FIG. 4 shows a rear plan view of the steamer with sanitization means, wherein the shroud is collapsed and attached to a handle of the steam unit via a retainer.

### DETAILED DESCRIPTION OF THE INVENTION

While this invention may be embodied in many different forms, there are described in detail herein specific embodiments of the invention. This description is exemplary of the principles of the invention and is not intended to limit the invention to the particular embodiments illustrated and described.

For the purpose of this disclosure, like reference numerals in the figures shall refer to like features unless otherwise indicated or is obvious by context.

The subject steamer with sanitization means is sometimes referred to as the device, the invention, the sanitizer, the machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation in the situation.

Referring now to the drawings, where the present invention is generally referred to with numeral 10, it can be observed that it basically includes a steam unit 11, a sanitization unit 20, and a shroud 30. The steam unit 11 is intended to retain and convert water to steam and generally comprises a reservoir 12, a handle 13, and a nozzle 14. Meanwhile, the sanitization unit 20 is intended to sterilize the fabric that is being steam cleaned by the steam unit 11. The sanitization unit 20 generally comprises a light assembly 21 and an actuation mechanism 22. The shroud 30 is used to direct both the steam and light onto a concentrated area of the fabric.

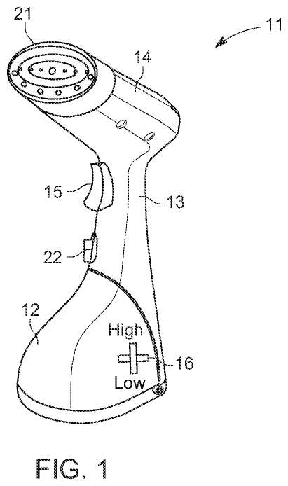

In reference to FIG. 1, the steam unit 11 is a hand-held device, wherein the nozzle 14 is in fluid communication with the reservoir 12 in order to dispense steam. The nozzle 14 is terminally connected to the handle 13, wherein the handle 13 is positioned in between the nozzle 14 and the reservoir 12. In some embodiments, the reservoir 12 may be terminally connected to the handle 13 opposite the nozzle 14.

The reservoir 12 can be either removably attached to the handle 13 or permanently affixed to the handle 13. When the reservoir 12 is removably attached to the handle 13, the reservoir 12 is detached from the handle 13 in order to fill the reservoir 12, wherein the reservoir 12 has a fill opening. The reservoir 12 may be attached to the handle 13 by a snap fit, threaded connection, spring lock, or any other suitable means for securing the reservoir 12 to the handle 13 in a temporary fashion.

When the reservoir 12 is permanently affixed to the handle 13, a fill cap is provided in addition to the fill opening. The fill cap is removably attached to the exterior of the reservoir 12, wherein the fill cap selectively opens and closes the fill opening for accessing or sealing the interior of the reservoir 12. The fill cap may be attached by a thread connection, snap connection, or any other suitable means. Alternatively, the handle 13 may comprise a fill channel, wherein the fill opening is in fluid communication with the fill channel. Furthermore, the fill cap is removably connected to the handle 13, wherein the fill cap is positioned adjacent to the fill channel opposite the fill opening. In this way, liquid passes through the handle 13 before entering the reservoir 12.

In other embodiments, the steam unit 11 further comprises a hose, wherein the hose is connected in between the reservoir 12 and the handle 13. In this way, the steam unit 11 is only partially hand-held. However, this allows for the reservoir 12 to be larger, thus reducing the frequency with which the reservoir 12 needs to be filled. Meanwhile, the nozzle 14 and handle 13 are still freely maneuverable due to the flexible nature of the hose. In such embodiments, the reservoir 12 may comprise one or more wheels as to make the reservoir 12 more portable across surfaces.

In some embodiments, the steam unit 11 may further comprise a trigger 15, wherein the trigger 15 is operably coupled to the reservoir 12, such that the actuation of the trigger 15 releases steam from the reservoir 12, thus allowing the steam to be dispensed through the nozzle 14. In some embodiments, the trigger 15 may be a pressure switch, or similar device, wherein the trigger 15 must be constantly engaged to release steam. Preferably the actuation mechanism 22 is integrated with the handle 13 in such embodiments, wherein the trigger 15 is engaged by one or more fingers of the user. When the user's finger or hand is removed from the trigger 15, the flow of steam to the nozzle 14 is cut off.

In other embodiments, the trigger 15 is a push button, flip switch, or similar mechanism, wherein the trigger 15 is engaged a first time to dispense steam and engaged a second time to terminate the flow of steam. In this way, the user does not need to maintain engagement of the trigger 15 as they operate the present invention. Furthermore, this allows the trigger 15 to be integrated with other parts of the steam unit 11, such as the nozzle 14 or reservoir 12.

In reference to FIG. 1, the sanitization unit 20 is integrated with the steam unit 11, wherein the light assembly 21 is configured to illuminate the fabric that is being steam cleaned. More specifically, the light assembly 21 is integrated with nozzle 14. The light assembly 21 is configured to emit sterilizing wavelengths of light, such as ultraviolet light. In the preferred embodiment, the light assembly 21 is configured to emit ultraviolet C light which is the best-known wavelength for effectively performing germicidal irradiation. However, different wavelengths may be used in other embodiments, as the different wavelengths may be found to be more effective depending on the material or the environment in which the device is placed.

In general, the light assembly 21 comprises a plurality of lighting elements, wherein the plurality of lighting elements is distributed around the opening of the nozzle 14. Preferably each of the plurality of lighting elements is a light emitting diode (LED), as LEDs lend to the compact, hand-held nature of the device. However, it is possible for each of the plurality of lighting elements to be any other suitable element capable of emitting irradiating light, such as an incandescent bulb, fluorescent bulb, or halogen bulb. The specific number of the plurality of lighting elements used depends on the type of light elements, the intensity of the light elements, and the placement of the light elements about the nozzle 14.

Alternatively, the light assembly 21 may be positioned on the leading edge or along the interior of the shroud 30. The light emitted will then be closer to the object being sanitized and may diffuse the light to even bathe the surface with the sanitizing light.

The light assembly 21 is electrically connected to the actuation mechanism 22, wherein the actuation mechanism 22 toggles the light assembly 21 between an on state and an off state. In the on state, the light assembly 21 emits ultraviolet C light, or another irradiating wavelength of light. The actuation mechanism 22 is integrated with the steam unit 11 such that the actuation mechanism 22 may be engaged by a user. Preferably the actuation mechanism 22 is integrated with the handle 13, such that the user may easily manipulate the actuation mechanism 22 with their finger as they hold the present invention. However, it is possible for the actuation mechanism 22 to be integrated with any other part of the steam unit, such as the nozzle 14 or reservoir 12.

In some embodiments, the actuation mechanism 22 is a push button, flip switch, or similar mechanism, wherein the actuation mechanism 22 is engaged a first time to switch the light assembly 21 to the on state and engaged a second time to switch the light assembly 21 to the off state. In other embodiments, the actuation mechanism 22 may be a pressure switch, or similar device, wherein the actuation mechanism 22 must be constantly engaged to maintain the light assembly 21 in the on state. Preferably the actuation mechanism 22 is integrated within the handle 13 in such embodiments, wherein the actuation mechanism 22 is engaged by the finger or hand of the user. When the finger or hand is removed, the actuation mechanism 22 is subsequently disengaged and the light assembly 21 is powered off.

In some embodiments, the actuation mechanism 22 is integrated with the trigger 15. In this way, only a single operating device is required to release steam and toggle the light assembly 21. Furthermore, this may reduce the profile of the present invention, increase the ease of operability for the user, and reduce manufacturing costs due to a reduced number of parts.

In yet other embodiments, the actuation mechanism 22 may be integrated with the nozzle 14, wherein the actuation mechanism 22 is engaged by the shroud 30. When the shroud 30 is attached to the nozzle 14, a portion of the shroud 30 engages the actuation mechanism 22, wherein the light assembly 21 is toggled to the on state. Conversely, when the shroud 30 is removed from the nozzle 14, the shroud 30 disengages the actuation mechanism 22, wherein the light assembly 21 is toggled to the off state.

In some embodiments, the sanitization unit 20 may further comprise a timer 23 to automatically switch the light assembly 21 from the on state to the off state. In this way, the user must only engage the actuation mechanism 22 a single time. Further, this limits the duration for which the light assembly 21 is switched to the on state and thus increases the energy efficiency of the present invention. The timer 23 is electrically connected with the light assembly 21 such that the timer 23 breaks the circuit after a preset time in order to toggle the light assembly 21 from the on state to the off state. In some embodiments the timer 23 may be integrated with the actuation mechanism 22, wherein the timer 23 flips the actuation mechanism 22 in order to break the circuit. In other embodiments, the timer 23 may have its own independent means for breaking the circuit.

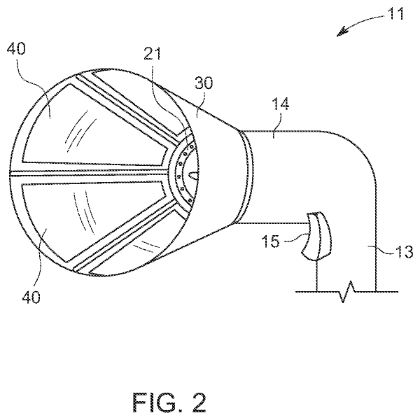

In reference to FIG. 2, the shroud 30 is attached to the nozzle 14, wherein the shroud 30 directs the steam and irradiating light onto a concentrated area of fabric. Preferably the shroud 30 is removable from the nozzle 14; however, in some embodiments, the shroud 30 may be permanently affixed to the nozzle 14. In some embodiments, the shroud 30 may taper towards the nozzle 14 forming a cone-like shape. In this way, the steam is away from the nozzle 14 in a larger radius, thus allowing for a larger area of concentrated steam. Additionally, the irradiating light is projected onto a larger area of fabric.

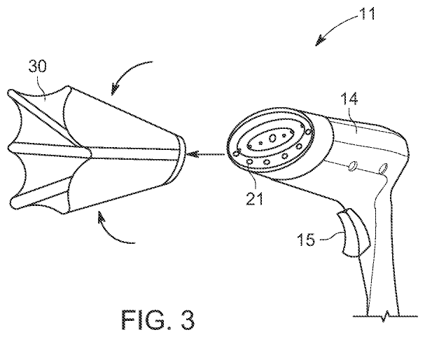

In reference to FIG. 3, in some embodiments, the shroud 30 is collapsible, such that when the shroud 30 is not in use it can be compacted in order to take up less space, thus being more travel and storage friendly. In such embodiments, the shroud 30 may comprise a plurality of linkages and a covering. Each of the plurality of linkages is distributed around the nozzle 14, while the covering is connected around the plurality of linkages. The covering is constructed from a flexible, non-breathable material. In this way, the covering is able to fold and compact as each of the plurality of linkages is collapsed; meanwhile, no steam is able to penetrate the covering which would reduce the effectiveness of directing the steam onto the fabric. When the shroud 30 is removed from the nozzle 14, each of the plurality of linkages may be folded similar to the linkages of an umbrella in order to reduce the profile of the shroud 30.

In other embodiments, the shroud 30 may be a telescoping member, such that the shroud 30 collapses in on itself. In such embodiments, the shroud 30 comprises a plurality of telescoping sections, wherein each telescoping section is nested within an adjacent section. Each of the plurality of telescoping sections is permanently connected to an adjacent section but in a manner such that the adjacent sections may slide along a common central axis. When the plurality of telescoping sections is extended outwards, each of the plurality of telescoping sections is temporarily locked in place. The plurality of telescoping sections may lock in place with a press fit, snap fit, twist lock, or any other suitable retaining means.

In yet other embodiments, the shroud 30 may comprise a plurality of removable sections, wherein each of the removable sections is temporarily attached to an adjacent section. The plurality of removable sections may lock in place with a press fit, snap fit, twist lock, or any other suitable retaining means. When the shroud 30 is in use, each of the plurality of removable sections is stacked in succession and locked in place. When the shroud 30 is no longer needed, each of the plurality of removable sections is unlocked and disconnected. Each of the plurality of removable sections may then be nested inside one another for storage.

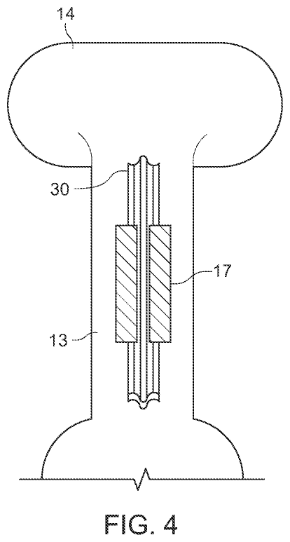

In reference to FIG. 4, the steam unit 11 may further comprise a retainer 17, wherein the retainer 17 is configured to receive the shroud 30. In this way, the retainer 17 allows the shroud 30 to be stored and transported alongside the steam unit 11. Preferably the retainer 17 is configured to receive the shroud 30 in the collapsed configuration; however, in some embodiments the retainer 17 may be configured to receive the shroud 30 in the extended position.

In some embodiments, the retainer 17 may be a clasp, clamp, or similar mechanism that engages with a portion of the shroud 30. As such, the shroud 30 is externally attached to the steam unit 11. In other embodiments, the retainer 17 is a slot formed into the steam unit 11. The slot may be partially formed into the steam unit 11, such as to create a channel into which the shroud 30 is positioned and stored external to the steam unit 11. One or more lips may be positioned along the channel, wherein the shroud 30 is snapped in and out of place. Alternatively, the slot may be a fully formed into the steam unit 11, such as to create a cavity within the steam unit 11 into which the shroud 30 is fully stowed.

In the preferred embodiment the shroud 30 is opaque, such that the irradiating light is not able to pass through the shroud 30. This allows the light assembly 21 to operate more efficiently and does not expose the user to the irradiating light. However, in some embodiments the shroud 30 may be transparent or semi-transparent. In this way, the user is able to readily differentiate when the light assembly 21 is in the on state and the off state to verify functionality.

In some embodiments, the shroud 30 may comprise the covering and a view port, wherein the covering is opaque and the view port is integrated with the covering. The view port allows the user to observe the status of the light assembly 21. More specifically, the view port allows the user to observe if the light assembly 21 is switched to the on state or the off state.

In some embodiments, the view port is formed by a grommet. In such an embodiment, the grommet reinforces the opening formed in the covering and provides a rigid or semi-rigid structure that maintains the hole in the covering in an open position, such that a user can view inside the shroud 30 as it is held against a fabric surface. The view port comprises a transparent sheet, wherein the grommet supports the transparent sheet about the hole in the covering. In this way, the shroud 30 remains fully enclosed but the user is still able to view the interior of the shroud 30. The transparent sheet may be fully transparent or semi-transparent, so long as enough light is able to pass through and allow the user to differentiate between the on state and the off state.

Furthermore, the transparent sheet may be rigid or flexible. The transparent sheet being rigid may lend to easier viewing of the interior of the shroud 30, as the transparent sheet would maintain the hole in the covering in an open position. On the other hand, the transparent sheet being flexible may lend to the shroud 30 being more compact, as it would be easier to fold, roll, or otherwise stow the view port portion along with the covering.

In reference to FIG. 2, the present invention may further comprise a reflective surface 40. The reflective surface 40 is integrated with the interior surface of the shroud 30, such that when the light assembly 21 is in the on state, the irradiating light is reflected about the interior of the shroud 30. In this way, the number of lighting elements of the light assembly 21 may be reduced, which in effect reduces energy consumption and may reduce manufacturing costs.

In addition to or in substitution to generating steam from water, the steam unit 11 may be configured to dispense chemicals or disinfectant solutions, including but not limited to isopropyl alcohol, zinc, or bleach (sodium hypochlorite). The steam unit 11 may be configured to heat the chemicals and dispense the chemicals as a steam. However, as it may be unsafe to heat some chemicals, the steam unit 11 may also be configured to dispense the chemicals in liquid form, such as in a spray. The chemicals may be dispensed from the same opening in the nozzle 14 as the steam or from a separate opening in the nozzle 14.

In such embodiments where chemicals are used, the reservoir 12 may comprise a first compartment and a second compartment. In this way, the water and the chemicals can be stored separate of one another. Alternatively, the steam unit 11 may comprise a subsequent reservoir 12, wherein the subsequent reservoir 12 contains the chemicals.

The nozzle 14 may comprise a throttle, wherein the throttle controls the amount of steam that is able to exit the opening of the nozzle 14. The throttle may be operated by a slide mechanism, twist mechanism, digital controls, or any other suitable means. In addition to varying the amount of steam that is able to exit the opening of the nozzle 14, the throttle may be used to adjust the spray pattern of the nozzle 14. In embodiments where chemicals are dispensed from a separate opening than that of the steam, the nozzle 14 may comprise a second throttle, wherein one throttle manipulates the flow of the steam and the other throttle manipulates the flow of the chemicals.

In reference to FIG. 1, the steam unit 11 may further comprise a power adjuster 16. The power adjuster 16 is used to toggle the rate at which steam is dispensed from the nozzle 14. In some embodiments, the power adjuster 16 may be a simple toggle switch which allows for set rates of steam to be selected. For example, the power adjuster 16 may allow the rate of steam to be toggled between low and high. In other embodiments, the power adjuster 16 may be a potentiometer, wherein the rate of steam may be fine-tuned. In yet other embodiments the rate of steam may be digitally controlled.

A version of the invention can be fairly described as a steamer with sanitization means comprising a steam unit further comprising a reservoir, a handle and a nozzle. The stem emitting nozzle is connected to the handle and directed forward. The handle is positioned between the nozzle and the reservoir and is dimensioned to be held by a human user. The nozzle is in fluid communication with the reservoir and a fluid in the reservoir is selectively heated into steam by a steam generator. The steam generator could be a heat producing element to heat the fluid (i.e. water, sterilizing mixture, fragrance or combination thereof) or a cool ultrasonic vaporizer or other similar technology to vaporize and expel the vapor or steam through the nozzle. Steam is intended to include any water vapor or vaporized fluid created by the device from the fluid in the reservoir. A sanitization unit is integrated with the steam unit and are used in concert. The sanitization unit comprising a sterilizing light assembly and an actuation mechanism, like a trigger or switch. The sterilizing light assembly is electrically connected to the actuation mechanism, wherein the sterilizing light assembly can be toggled between an on state and an off state. The sterilizing light assembly is integrated to the steam unit about the nozzle. The nozzle is configured to emit steam and the sterilizing light assembly is configured to emit ultraviolet light (or other wavelength of light having sterilizing characteristics) in the on state. A shroud is selectively engaged around the nozzle. The shroud is positioned around the sterilizing light assembly. Optionally, the shroud collapses. Optionally, the steam unit further comprises a trigger as a switch where the trigger is integrated with the handle; and the trigger is operably coupled to both the light assembly and the steam generator to turn both on simultaneously or either individually selectively. Optionally, the steam unit further comprises a retainer that is configured to receive and affix to the shroud. Optionally, a reflective surface is positioned about an interior surface of the shroud. Optionally, an interior surface of the shroud includes a second sterilizing light assembly.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

I claim:

1. A steamer with sanitization means comprising:
a steam unit comprising a reservoir, a handle and a nozzle;
the nozzle is connected to the handle;
the handle is positioned between the nozzle and the reservoir;
the nozzle is in fluid communication with the reservoir;
a fluid in the reservoir is selectively heated into steam by a steam generator;
a sanitization unit is integrated with the steam unit;
the sanitization unit comprising a sterilizing light assembly and an actuation mechanism;
the light assembly is electrically connected to the actuation mechanism, wherein the sterilizing light assembly can be toggled between an on state and an off state;
the sterilizing light assembly is integrated to the steam unit about the nozzle;
the nozzle is configured to emit steam and the sterilizing light assembly is configured to emit ultraviolet light in the on state;
a shroud is selectively engaged around the nozzle;
the shroud is positioned around the light assembly.

2. The steamer with sanitization means in claim 1, wherein the shroud collapses.

3. The steamer with sanitization device in claim 1 further characterized in that the steam unit further comprises a trigger;
the trigger is integrated with the handle; and
the trigger is operably coupled to both the sterilizing light assembly and the steam generator.

4. The steamer with sanitization means as claimed in claim 3, wherein the actuation mechanism is integrated with the trigger.

5. The steamer with sanitization means as claimed in claim 1 further characterized in that:
the steam unit further comprising a retainer;
the retainer is configured to receive and affix to the shroud.

6. The steamer with sanitization means as claimed in claim 1 further characterized in that a reflective surface is positioned about an interior surface of the shroud.

7. The steamer with sanitization means as claimed in claim 1 further characterized in that an interior surface of the shroud includes a second sterilizing light assembly.

8. The steamer with sanitization means as claimed in claim 3 further characterized in that an interior surface of the shroud includes a second sterilizing light assembly.

9. The steamer with sanitization means as claimed in claim 5 further characterized in that an interior surface of the shroud includes a second sterilizing light assembly.

10. The steamer with sanitization means as claimed in claim 6 further characterized in that an interior surface of the shroud includes a second sterilizing light assembly.
